(19) United States
(12) Patent Application Publication
(10) Pub. No.: US 2020/0196939 A1
(43) Pub. Date: Jun. 25, 2020

(54) Monthly Cycle Fitness Optimizer

(71) Applicant: Arri Russell Morris, Austin, TX (US)

(72) Inventor: Arri Russell Morris, Austin, TX (US)

(21) Appl. No.: 16/231,511

(22) Filed: Dec. 23, 2018

(51) Int. Cl. A61B 5/00; A61B 5/0205; A61B 5/01; A61B 5/026; A61B 10/00; G01N 33/74; G01N 33/76; G09B 19/00; G16H 20/60

(52) U.S. Cl. CPC A61B 5/486; A61B 5/02055; A61B 5/01; A61B 5/026; A61B 10/0012; A61B 5/024; G01N 33/743; G01N 33/76; G09B 19/003; G16H 20/60; A61B 5/7264

(57) ABSTRACT

Described herein are various principles related to collecting and analyzing fertility data for female humans. The underlying concept is that a woman's hormones fluctuate throughout the menstrual cycle, affecting optimal exercise routines and general health practices. A dedicated sensor may be used to collect fertility data, or an estimate may be derived from the individual's menstrual history. Once collected or estimated, the fertility data is factored with other variables to determine the optimal exercise routine or general health habits for the woman. The recommendations are communicated to the woman, who may provide feedback to further improve future recommendations.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### RELATED APPLICATIONS

| | | |
|---|---|---|
| 20160174946 | June 2016 | Sacks et al. |
| 20170039336 | February 2017 | Bitran et al. |
| 10/068,494 | September 2018 | Ahmad et al. |

### BACKGROUND

The menstrual cycle is a natural monthly event for many women. The cycle affects key hormones that regulate fertility and metabolism, altering the impact of physical activity. In terms of physical performance, it has been reported that women in the Luteal Phase take longer to become exhausted. However, this does not necessarily mean that all women should increase exercise intensity during the Luteal Phase.

Depending on an individual woman's goal, the optimal strategy for navigating her body's hormonal fluctuations can vary greatly. If she wants to gain muscle, lose fat, or increase endurance, the effects of estrogen, progesterone, and other hormones should be weighed differently for each case.

Although fertility trackers are commercially available and fitness trackers have gained popularity, no systems have been marketed to guide women's exercise based upon the menstrual cycle.

Patents referenced in this application represent two separate, related technologies (2016/0174946, 2017/0039336) and a similar, combined technology (10068494). However, the system designed by Ahmad, et al. bases its recommendations on ketone levels, not hormones.

### SUMMARY

There is provided a method for identifying optimal exercise routines for women. The method comprises receiving the woman's personal goals, history of the menstrual cycle, and an estimate of her progression in the current cycle. The estimate can comprise of biometric data from a device, such as basal body temperature, or temporal data provided directly by the user, such as average cycle length and the last known date of menstruation. The goals are weighed formulaically against known effects of hormones and relative levels of each hormone during a given day of the menstrual cycle to produce an optimized exercise routine.

### BRIEF DESCRIPTION OF DRAWINGS

FIG. 1 is the process flow when using a biometric sensor

FIG. 2 is the process flow when no sensor is used

FIG. 3 is an expanded view of the software process flow

FIG. 4 charts relative concentrations of primary sex hormones during the menstrual cycle

### DETAILED DESCRIPTION

Both paper templates and electronic apparatuses exist for people to chart collected data regarding fertility characteristics. Commercially available tools are becoming increasingly convenient. While fitness tracking and advisory technology has become increasingly convenient as well, a link between the two technologies has not been explicitly established. A combination of these technologies could lead an evolution in the market from fitness trackers to fitness trainers. Using fertility data, a woman could more efficiently apply her energy towards exercises that bring her closer to her personal goals.

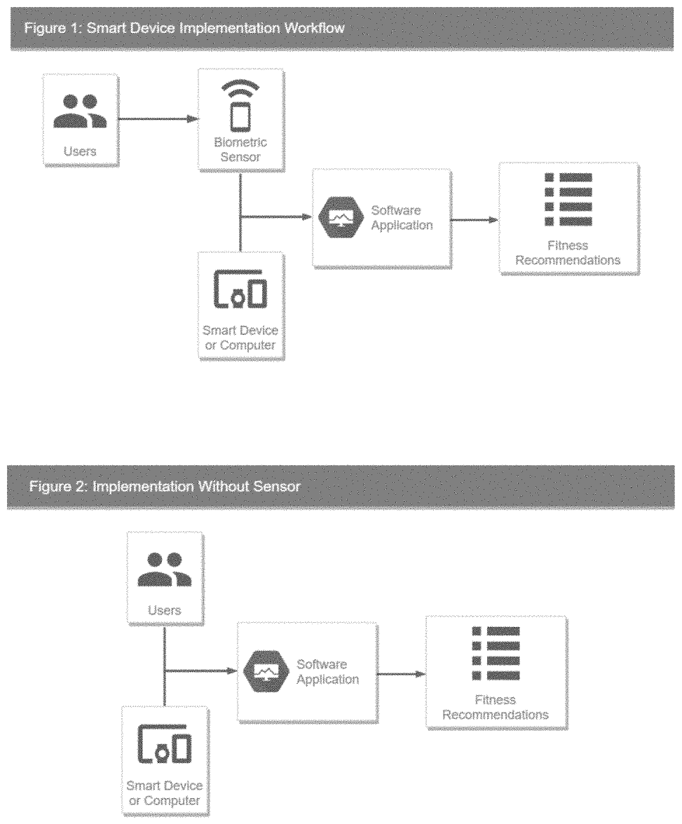

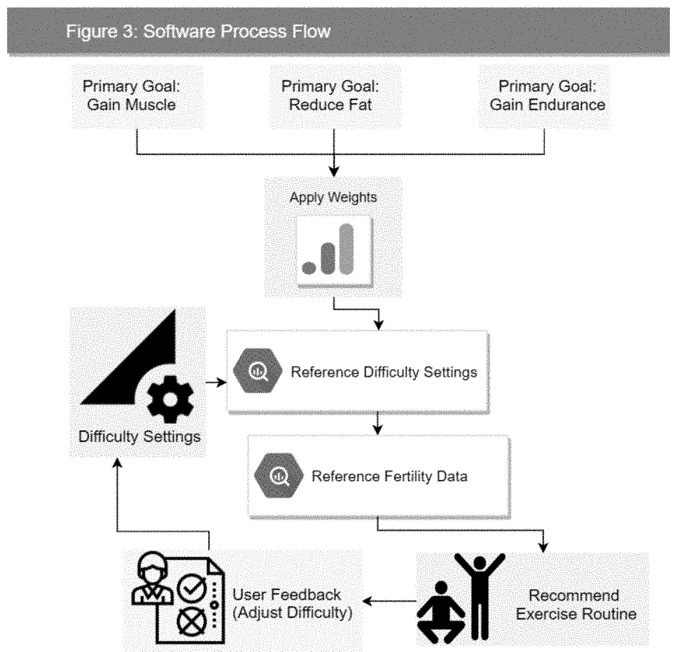

In view of the aforementioned, described herein are various embodiments of the core principle under consideration: using fertility data to recommend exercise routines and general health practices. In some embodiments, a user may collect fertility data through biometric sensors as depicted in FIG. 1. In other embodiments, an estimation of the current menstrual cycle may be applied from the user's history and average characteristics of the menstrual cycle as depicted in FIG. 2. Although much of the process is expected to occur in a software application, the core principle is simple enough to replicate via a paper form.

The difference between the overall process in FIG. 1, and FIG. 2 is convenience and cost of the underlying system. While FIG. 1 illustrates the process implemented wherein the user would be required to enter less information regarding her monthly cycle, the process implemented in FIG. 2 does not require a fertility tracking device. In both FIG. 1 and FIG. 2, the software application referenced is further illustrated by FIG. 3.

In FIG. 3 the user selects her primary goal, having already provided her current menstrual cycle information either via a fertility tracking device or by entering dates of menstruation. Although three options are given in this example, they should not be considered limiting, as more may be deemed necessary to address market demand.

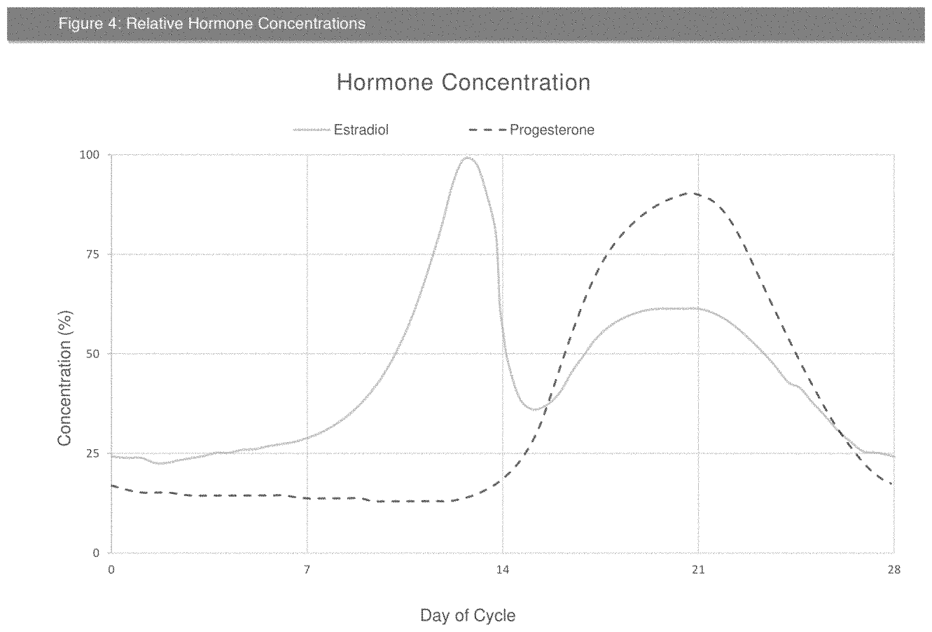

Depending on the goal selected, mathematical weights will be applied to a pool of potential exercise activities. These activities are first checked against the requested difficulty of the workout or health practice, and then cross-referenced by the effectiveness for a particular day. The curves in FIG. 4 representing relative concentrations of hormones for a given day in the cycle are used to estimate the effectiveness of a given activity or health practice. For example, if the goal is to reduce fat, the user should conserve her energy during days 18-25 of the cycle, when progesterone is highest, by doing moderate exercise.

FIG. 4 assumes a typical 4-week cycle, however the curves may be compressed or expanded depending on a user's average cycle length. Though not currently widely available, these assumptions may be overwritten by a biometric device that directly tracks hormone levels. After the user has completed the recommended activity or acknowledged the suggested health practice, she will be given an opportunity to rate the difficulty of the routine or suggestion on a 5-point scale from so that a machine learning algorithm may further refine future recommendations.

An example of this algorithmic refinement is as follows:
1. The routine recommended for the day is 5 minutes of jumping rope, 25 lunges, 30 bear-crawls and 12 pushups. These activities had underlying difficulty scores of 5, 5, 6, and 4 respectively, for a total of 20, the default difficulty level for beginners.
2. When asked for feedback, the user rates the activity as “A little too easy” on the 5 point scale.
3. The underlying difficulty rating for each activity is reduced by 10%.
4. The next day, the same activities are recommended as they are determined to still be the most efficient activities for the primary goal.
5. To meet the minimum difficulty threshold of 20, 6 minutes of jumping rope, 28 lunges, 33 bear crawls, and 13 pushups are recommended.
6. The user rates the revised routine “Just right”.

Having thus described several aspects of embodiments, it should be understood that various alterations, modifications, and improvements will readily occur to those skilled in the art. The specific routines or methods described herein may represent one or more of any number of processing strategies. In particular, the machine learning algorithm presented performs the basic function that is claimed, but will likely be further developed.

Various acts illustrated or described may be performed in the sequence illustrated or described, in other sequences, in parallel, or omitted. Likewise, the order of the above-described processes may be changed. Accordingly, the foregoing description and drawings are by way of example only.

1. A method of using women's monthly cycle for recommending fitness routines or general health coaching, the method comprising: receiving fertility data, the fertility data comprising information used to pinpoint or estimate the current progression of the menstrual cycle and the relative concentration of hormones; weighing the effects of menstrual hormones as a factor when determining recommendations or general health coaching; and recommending exercise routines or coaching the user on general health practices.

2. The method of claim 1, wherein the woman's menstrual cycle is tracked or estimated with biometric measurements related to the cycle such as, but not limited to, basal body temperature, menstruation dates, resting pulse rate, bioimpedance, breathing rate, perfusion, and levels of hormones such as: progesterone, estradiol, follicle stimulating hormone, luteinizing hormone, or any combination thereof.

3. The method of claim 1, which further optimizes fitness routines or general health coaching with artificial intelligence, including machine learning algorithms.
